(19) United States
(12) Patent Application Publication
(10) Pub. No.: US 2026/0159473 A1
(43) Pub. Date: Jun. 11, 2026

(54) DEVICE AND METHOD FOR CONTINUOUS RECYCLING OF ORGANIC BASE

(71) Applicants: Fudan University, Shanghai (CN); Jiangxi Normal University, Nanchang (CN)

(72) Inventors: Fener CHEN, Shanghai (CN); Xianjing ZHENG, Shanghai (CN); Chang LIU, Shanghai (CN); Yiqun TIAN, Shanghai (CN); Yajiao ZHANG, Shanghai (CN); Li WAN, Shanghai (CN); Chunxue PENG, Shanghai (CN); Songlin ZHENG, Shanghai (CN); Liangchuan LAI, Shanghai (CN)

(21) Appl. No.: 19/181,287

(22) Filed: Apr. 16, 2025

(30) Foreign Application Priority Data
Apr. 22, 2024 (CN) 202410483420.3

(51) Int. Cl. C07C 209/84; C07C 209/86; C07C 211/05

(52) U.S. Cl. CPC C07C 209/84; C07C 209/86; C07C 211/05

(57) ABSTRACT

A continuous recycling device of an organic base, including a first pump, a second pump, a third pump, a micro-mixer, a micro-channel reactor, a liquid-liquid separator and a continuous stirred tank. The third pump is configured to feed a first base-containing waste liquid to the micro-mixer. The first pump is configured to feed a solution of a second base to the micro-mixer. The micro-mixer is configured to mix the first base-containing waste liquid with the solution of the second base, and transfer the mixture to the micro-channel reactor. The liquid-liquid separator is configured to divide the reaction mixture into a top layer and a bottom layer. The continuous stirred tank is filled with a water-absorbing agent to absorb water in the top layer. A continuous recycling method using such device is also provided.

### CROSS-REFERENCE TO RELATED APPLICATIONS

This application claims the benefit of priority from Chinese Patent Application No. 202410483420.3, filed on Apr. 22, 2024. The content of the aforementioned application, including any intervening amendments thereto, is incorporated herein by reference in its entirety.

### TECHNICAL FIELD

This application relates to fine chemical technologies, and more particularly to a device and method for continuous recycling of an organic base.

### BACKGROUND

Organic base is often used as a catalyst or an acid-binding agent in the condensation reaction and substitution reaction, and is greatly consumed in the synthesis reaction. The organic base often forms complex salts with hydrochloric acid, sulfuric acid and phosphoric acid after reaction, and the resultant complex salts will be dissolved in water during the post-extraction treatment, thereby producing a large amount of waste liquid containing organic base. The treatment of the organic base-containing waste liquid involves complex operation and high cost. Therefore, in order to reduce the chemical waste discharge and lower the production cost, it is necessary to develop a strategy to achieve efficient extraction, recovery and reuse of organic base from the organic base-containing waste liquid.

Several method and devices have been developed for recycling triethylamine (e.g., Chinese Patent Nos. 218951289U, 103304423B and 208104264U and Chinese Patent Publication), but they struggle with use of many tanks for storage and standing, large space occupation and poor recycling efficiency. Optionally, an azeotropic distillation column may be adopted to realize continuous recycling for triethylamine, which will result in large energy consumption and high recycling costs. Therefore, it is urgent to develop a new system for efficiently recycling the organic base from the waste liquid with optimized recovery rate, simplified operation process, improved purity, and lowered safety risks, so as to arrive at the environmentally-friendly and cost-effective treatment of the organic base-containing waste liquid.

### SUMMARY

In view of the problems in the prior art, this application provides a device and method for continuous recycling of an organic base, which has high recovery efficiency and low operating costs.

Regarding the device and method provided herein for continuous recycling of an organic base, feed pumps, micro-mixers, micro-channel reactors, liquid-liquid separators and continuous kettles conforming to reaction characteristics are adopted and connected sequentially, leading to high recovery rate of base (greater than 99%) and high recovery purity. The recovered base can be directly reused without additional treatment. This application has high integration degree, small space occupation and excellent operation stability, avoiding the defects in the traditional batch process (e.g., arrangement of many tanks for storage and standing, large space occupation and poor recovery efficiency).

This application provides a method for continuous recycling of an organic base using a continuous recycling device, the continuous recycling device comprising a first pump, a second pump, a third pump, a micro-mixer, a micro-channel reactor, a liquid-liquid separator and a continuous stirred tank; the first pump, the micro-mixer, the micro-channel reactor, the liquid-liquid separator, the continuous stirred tank and the second pump being connected sequentially; the third pump being communicated with the micro-mixer; the continuous stirred tank being equipped with a heat exchanger and filled with a water-absorbing agent; and
the continuous recycling method comprises:
(S1) reacting a reaction material and a first base followed by post-treatment to collect a waste liquid containing the first base;
transporting, by the third pump, the waste liquid to the micro-mixer; transporting, by the first pump, a solution of a second base to the micro-mixer; mixing the waste liquid with the solution of the second base in the micro-mixer to obtain a mixture; subjecting the mixture to alkalization reaction in the micro-channel reactor at 0-90° C. for 0.1-30 min to obtain an alkalified solution; and
(S2) transferring the alkalified solution from the micro-channel reactor to the liquid-liquid separator, followed by division into a top layer and a bottom layer under the action of gravity, wherein the bottom layer is an aqueous solution of the second base, and the top layer is a mixture of the first base and water; concentrating the bottom layer, and collecting a concentrated product as the solution of the second base for use; and
transferring the top layer from the liquid-liquid separator to the continuous stirred tank followed by stirring to allow the water in the top layer to be absorbed by the water-absorbing agent; and pumping, by the second pump, the first base from a material outlet at an upper part of the continuous stirred tank to a storage tank to achieve recovery of the first base.

In an embodiment, the first pump, the second pump and the third pump are each a plunger pump for liquid transfer.

In an embodiment, the micro-mixer is specially designed, and is a composite plate-type micro-mixer composed of 15-18 rhombic microchannel mixing components connected in series; each of the rhombic microchannel mixing components is provided with a first fluid channel with a round cross section with a diameter of 100 μm-20 mm or a square cross section with a side length of 100 μm-20 mm, a length of 1-100 cm, and an applicable flux of 1-3000 mL/min; in an embodiment, the first fluid channel of each of the rhombic microchannel mixing components has a round cross section with a diameter of 1-20 mm, a length of 10-100 cm, and an applicable flux of 1000-3000 mL/min; or the first fluid channel of each of the rhombic microchannel mixing components has a square cross section with a side length of 1-20 mm, a length of 10-100 cm, and an applicable flux of 1000-3000 mL/min; a material of the first fluid channel of each of the rhombic microchannel mixing components is selected from the group consisting of glass, polytetrafluoroethylene, stainless steel, hastelloy, tantalum material, zirconium material and a combination thereof.

In an embodiment, the micro-channel reactor is specially designed, and is a tubular micro-reactor equipped with a static mixing component; a main body of the micro-channel reactor is a tubular cavity, and is configured as a second fluid channel; a first end of the tubular cavity is provided with a reactant material inlet, and a second end of the tubular cavity is provided with a reactant material outlet; a plurality of components each in an double-cross shape are axially arranged in the tubular cavity; a first heat-exchanging fluid interlayer is provided outside the tubular cavity for a heat-exchanging fluid to pass through; a first end of the first heat-exchanging fluid interlayer is provided with a first heat-exchanging fluid outlet, and a second end of the first heat-exchanging fluid interlayer is provided with a first heat-exchanging fluid inlet; the tubular cavity has a diameter of 100 μm-20 mm and a length of 1 m-5000 m; in an embodiment, the tubular cavity has a diameter of 5 mm-20 mm and a length of 1 m-5000 m; and a material of the tubular cavity is selected from the group consisting of glass, polytetrafluoroethylene, stainless steel, hastelloy, tantalum material, zirconium material and a combination thereof.

In an embodiment, the liquid-liquid separator is designed according to a gravitational settling principle; a main body of the liquid-liquid separator is a cylindrical cavity, which is vertically arranged; a lower end of the cylindrical cavity is provided with an inlet for the alkalified solution, and is also provided with an outlet of the bottom layer; an upper end of the cylindrical cavity is provided with an outlet of the top layer; an outer side of the cylindrical cavity is provided with a second heat-exchanging fluid interlayer for the heat-exchanging fluid to pass through; a lower part of the second heat-exchanging fluid interlayer is provided with a second heat-exchanging fluid inlet, and an upper part of the second heat-exchanging fluid interlayer is provided with a second heat-exchanging fluid outlet; the liquid-liquid separator has an inner diameter of 1-20 cm and a height of 1-200 cm; and a material of the liquid-liquid separator is selected from the group consisting of glass, polytetrafluoroethylene, stainless steel, hastelloy, tantalum material, zirconium material and a combination thereof.

In an embodiment, the continuous stirred tank is a cylinder structure, and is vertically arranged; an interior of the continuous stirred tank is provided with a stirring blade, and an outer side of the continuous stirred tank is provided with a heat-exchanging jacket; a lower part of the continuous stirred tank is provided with a material inlet, and an upper part of the continuous stirred tank is provided with a material outlet; a lower part of the heat-exchanging jacket is provided with a third heat-exchanging fluid inlet, and an upper part of the heat-exchanging jacket is provided with a third heat-exchanging fluid outlet; the continuous stirred tank is filled with the water-absorbing agent; the material inlet is in pipeline connection with the liquid-liquid separator, and the material outlet is in pipeline connection with the second pump; the continuous stirred tank has an inner diameter of 5-1000 cm and a height of 5-1000 cm; and a material of the continuous stirred tank is selected from the group consisting of glass, polytetrafluoroethylene, stainless steel, hastelloy, tantalum material, zirconium material and a combination thereof.

In an embodiment, the first base is liquid ammonia, triethylamine, trimethylamine, tributylamine, diethylamine or N, N-diisopropylethylamine;
a second base is potassium carbonate, sodium carbonate, sodium bicarbonate, sodium hydroxide, potassium hydroxide or lithium hydroxide; and
the water-absorbing agent is selected from the group consisting of potassium carbonate, sodium carbonate, sodium bicarbonate, sodium hydroxide, potassium hydroxide, lithium hydroxide, sodium sulfate, magnesium sulfate, molecular sieve and a combination thereof.

In an embodiment, the micro-channel reactor has a temperature of 0-90° C. and a residence time of 0.1-30 min; preferably, the micro-channel reactor has a temperature of 20-60° C. and a residence time of 5-20 min.

This application also provides a continuous recycling device for an organic base, comprising a first pump, a second pump, a third pump, a micro-mixer, a micro-channel reactor, a liquid-liquid separator and a continuous stirred tank; wherein the micro-mixer, the micro-channel reactor, the liquid-liquid separator and the continuous stirred tank are connected sequentially through a pipeline; the first pump and the third pump are connected to the micro-mixer; the second pump is connected to the continuous stirred tank, and is configured to pump the first base from the continuous stirred tank to a first storage tank; the first pump is configured to pump a solution of the second base to the micro-mixer; the third pump is configured to pump a waste liquid containing the first base from a second storage tank to the micro-mixer; the micro-mixer is configured to mix the first base with the solution of the second base to obtain a mixture, and transfer the mixture to the micro-channel reactor for alkalization to obtain an alkalified solution; the liquid-liquid separator is configured to separate the alkalified solution into a top layer and a bottom layer; a lower part of the liquid-liquid separator is provided with a first outlet for discharging the bottom layer; and an upper part of the liquid-liquid separator is provided with a second outlet for discharging the top layer to the continuous stirred tank; the bottom layer discharging from the first outlet is concentrated and then transferred to a storage pool for reuse; the top layer is treated by the continuous stirred tank to remove the water in the top layer, followed by being pumped by the second pump; the top layer is subjected to a series of treatments, and is transferred to the first storage tank; and such processes are cycled.

### BRIEF DESCRIPTION OF THE DRAWINGS

FIG. 1 is a flow chart of a continuous recycling device and method for an organic base according to an embodiment of the present disclosure.

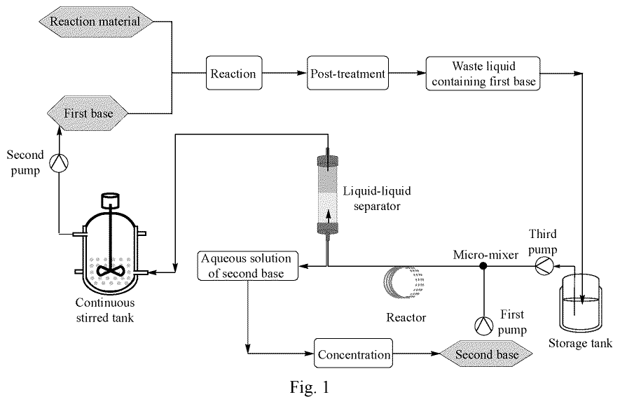

FIG. 2 is a structural diagram of plate-type micro-mixers in zigzag series according to an embodiment of the present disclosure.

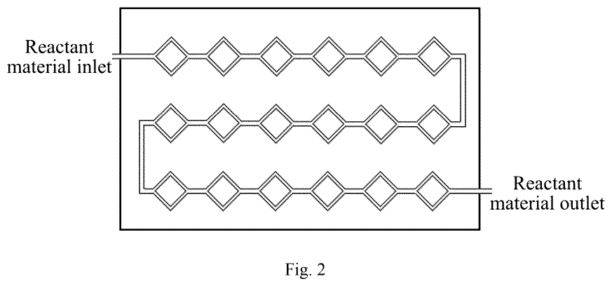

FIG. 3 is a structural diagram of a tubular micro-reactor internally provided with double-cross shape mixing components according to an embodiment of the present disclosure.

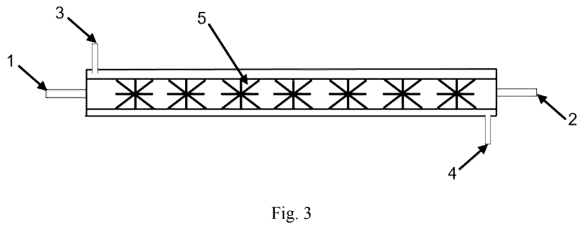

FIG. 4 is a structural diagram of a liquid-liquid separator according to an embodiment of the present disclosure.

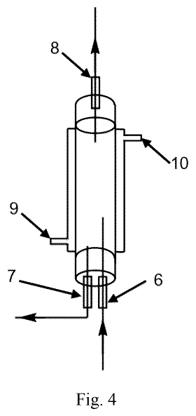

FIG. 5 is a structural diagram of a continuous stirred tank according to an embodiment of the present disclosure.

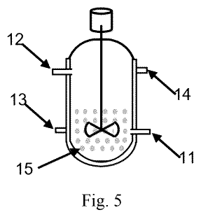

In Figures: 1, reactant material inlet; 2, reactant material outlet; 3, first heat-exchanging fluid outlet; 4, first heat-exchanging fluid inlet; 5, double-cross shape mixing component; 6, inlet for an alkalified solution; 7, outlet of the bottom layer; 8, outlet of the top layer; 9, second heat-exchanging fluid inlet; 10, second heat-exchanging fluid outlet; 11, material inlet; 12, material outlet; 13, third heat-exchanging fluid inlet; 14, third heat-exchanging fluid outlet; and 15, water-absorbing agent.

### DETAILED DESCRIPTION OF EMBODIMENTS

The present disclosure is further described through embodiments with reference of accompanying drawings.

### Example 1

A reaction material and a first base trimethylamine were reacted followed by post-treatment to collect a trimethylamine salt waste liquid. The trimethylamine salt waste liquid and a 20% sodium hydroxide solution pumped by a first pump were mixed in a micro-mixer, and then were transferred to a micro-channel reactor for alkalization to obtain an alkalified solution, where the micro-channel reactor had a residence time of 5 min and a temperature of 40° C. The alkalified solution was directly transferred to a liquid-liquid separator for rapid stratification under the action of gravity, where a bottom layer was a sodium hydroxide aqueous solution, and a top layer was a trimethylamine solution contained a small amount of water. The sodium hydroxide aqueous solution was concentrated to obtain the 20% sodium hydroxide solution for reuse. The trimethylamine solution contained a small amount of water was directly transferred to a continuous stirred tank with stirring and heat-exchanging functions. The continuous stirred tank filled with anhydrous sodium sulfate which was used as a water-absorbing agent. After stirring, water in the trimethylamine solution was removed to obtain the trimethylamine, and the trimethylamine was directly pumped, by a second pump connected with a material outlet through a pipeline, into a storage tank, so as to realize recycle. The trimethylamine had a recovery rate of 95.1%, a purity of 99% and a water content less than 0.1%.

### Example 2

A reaction material and a first base triethylamine were reacted followed by post-treatment to collect a triethylamine salt waste liquid. The triethylamine salt waste liquid and a 20% potassium hydroxide solution pumped by a first pump were mixed in a micro-mixer, and then were transferred to a micro-channel reactor for alkalization to obtain an alkalified solution, where the micro-channel reactor had a residence time of 3 min and a temperature of 40° C. The alkalified solution was directly transferred to a liquid-liquid separator for rapid stratification under the action of gravity, where a bottom layer was a potassium hydroxide aqueous solution, and a top layer was a triethylamine solution contained a small amount of water. The potassium hydroxide aqueous solution was concentrated to obtain the 20% potassium hydroxide solution for reuse. The triethylamine solution contained a small amount of water was directly transferred to a continuous stirred tank with stirring and heat-exchanging functions. The continuous stirred tank filled with anhydrous sodium sulfate which was used as a water-absorbing agent. After stirring, water in the triethylamine solution was removed to obtain the triethylamine, and the triethylamine was directly pumped, by a second pump connected with a material outlet through a pipeline, into a storage tank, so as to realize recycle. The triethylamine had a recovery rate of 96.3%, a purity of 99% and a water content less than 0.1%.

### Example 3

A reaction material and a first base triethylamine were reacted followed by post-treatment to collect a triethylamine salt waste liquid. The triethylamine salt waste liquid and a 20% potassium hydroxide solution pumped by a first pump were mixed in a micro-mixer, and then were transferred to a micro-channel reactor for alkalization to obtain an alkalified solution, where the micro-channel reactor had a residence time of 3 min and a temperature of 40° C. The alkalified solution was directly transferred to a liquid-liquid separator for rapid stratification under the action of gravity, where a bottom layer was a potassium hydroxide aqueous solution, and a top layer was a triethylamine solution contained a small amount of water. The potassium hydroxide aqueous solution was concentrated to obtain the 20% potassium hydroxide solution for reuse. The triethylamine solution contained a small amount of water was directly transferred to a continuous stirred tank with stirring and heat-exchanging functions. The continuous stirred tank filled with anhydrous potassium hydroxide which was used as a water-absorbing agent. After stirring, water in the triethylamine solution was removed to obtain the triethylamine, and the triethylamine was directly pumped, by a second pump connected with a material outlet through a pipeline, into a storage tank, so as to realize recycle. The triethylamine had a recovery rate of 99.1%, a purity of 99% and a water content less than 0.1%.

### Example 4

A reaction material and a first base triethylamine were reacted followed by post-treatment to collect a triethylamine salt waste liquid. The triethylamine salt waste liquid and a 25% sodium hydroxide solution pumped by a first pump were mixed in a micro-mixer, and then were transferred to a micro-channel reactor for alkalization to obtain an alkalified solution, where the micro-channel reactor had a residence time of 4 min and a temperature of 35° C. The alkalified solution was directly transferred to a liquid-liquid separator for rapid stratification under the action of gravity, where a bottom layer was a sodium hydroxide aqueous solution, and a top layer was a triethylamine solution contained a small amount of water. The sodium hydroxide aqueous solution was concentrated to obtain the 25% sodium hydroxide solution for reuse. The triethylamine solution contained a small amount of water was directly transferred to a continuous stirred tank with stirring and heat-exchanging functions. The continuous stirred tank filled with anhydrous sodium hydroxide which was used as a water-absorbing agent. After stirring, water in the triethylamine solution was removed to obtain the triethylamine, and the triethylamine was directly pumped, by a second pump connected with a material outlet through a pipeline, into a storage tank, so as to realize recycle. The triethylamine had a recovery rate of 99.6%, a purity of 99% and a water content less than 0.1%.

### Example 5

A reaction material and a first base triethylamine were reacted followed by post-treatment to collect a triethylamine salt waste liquid. The triethylamine salt waste liquid and a 25% sodium hydroxide solution pumped by a first pump were mixed in a micro-mixer, and then were transferred to a micro-channel reactor for alkalization to obtain an alkalified solution, where the micro-channel reactor had a residence time of 4 min and a temperature of 35° C. The alkalified solution was directly transferred to a liquid-liquid separator for rapid stratification under the action of gravity, where a bottom layer was a sodium hydroxide aqueous solution, and a top layer was a triethylamine solution contained a small amount of water. The sodium hydroxide aqueous solution was concentrated to obtain the 25% sodium hydroxide solution for reuse. The triethylamine solution contained a small amount of water was directly transferred to a continuous stirred tank with stirring and heat-exchanging functions. The continuous stirred tank filled with anhydrous sodium hydroxide which was used as a water-absorbing agent. After stirring, water in the triethylamine solution was removed to obtain the triethylamine, and the triethylamine was directly pumped, by a second pump connected with a material outlet through a pipeline, into a storage tank, so as to realize recycle. The triethylamine had a recovery rate of 99.8%, a purity of 99% and a water content less than 0.1%.

It should be noted that the above description is merely illustrative, and is not intended to limit the present disclosure. It should be understood that various changes, modifications and replacements made to the technical features recited in the embodiments without departing from the spirit of the disclosure shall fall within the scope of this application defined by the appended claims.

What is claimed is:

1. A method for continuous recycling of an organic base using a continuous recycling device, the continuous recycling device comprising a first pump, a second pump, a third pump, a micro-mixer, a micro-channel reactor, a liquid-liquid separator and a continuous stirred tank; the first pump, the micro-mixer, the micro-channel reactor, the liquid-liquid separator, the continuous stirred tank and the second pump being connected sequentially; the third pump being communicated with the micro-mixer;
the continuous stirred tank being equipped with a heat exchanger and filled with a water-absorbing agent; and the method comprising:
(S1) reacting a reaction material with a first base followed by post-treatment to collect a waste liquid containing the first base;
transporting, by the third pump, the waste liquid to the micro-mixer; transporting, by the first pump, a solution of a second base to the micro-mixer; mixing the waste liquid with the solution of the second base in the micro-mixer to obtain a mixture;
subjecting the mixture to alkalization reaction in the micro-channel reactor at 0 -90° C. for 0.1-30 min to obtain an alkalified solution; and
(S2) transferring the alkalified solution from the micro-channel reactor to the liquid-liquid separator, followed by division into a top layer and a bottom layer under the action of gravity, wherein the bottom layer is an aqueous solution of the second base, and the top layer is a mixture of the first base and water; concentrating the bottom layer, and collecting a concentrated product as the solution of the second base for use; and
transferring the top layer from the liquid-liquid separator to the continuous stirred tank followed by stirring to allow the water in the top layer to be absorbed by the water-absorbing agent; and pumping, by the second pump, the first base from the continuous stirred tank to a storage tank to achieve recovery of the first base.

2. The method of claim 1, wherein the first pump, the second pump and the third pump are each a plunger pump for liquid transfer.

3. The method of claim 1, wherein the micro-mixer is a composite plate-type micro-mixer composed of 15-18 rhombic microchannel mixing components connected in series; a fluid channel of each of the 15-18 rhombic microchannel mixing components has a round cross section with a diameter of 100 μm-20 mm or a square cross section with a side length of 100 μm-20 mm, a length of 1-100 cm, and an applicable flow of 1-3000 mL/min.

4. The method of claim 1, wherein the micro-channel reactor is a tubular micro-reactor equipped with a static mixing component; a main body of the micro-channel reactor is a tubular cavity, and is configured as a fluid channel; a first end of the tubular cavity is provided with a reaction material inlet, and a second end of the tubular cavity is provided with a reaction material outlet; a plurality of components each in an double-cross shape are axially arranged in the tubular cavity; a heat-exchanging interlayer is provided outside the tubular cavity for a heat-exchanging fluid to pass through; a first end of the heat-exchanging interlayer is provided with a heat-exchanging fluid outlet, and a second end of the heat-exchanging interlayer is provided with a heat-exchanging fluid inlet; and the tubular cavity has a diameter of 100 μm-20 mm and a length of 1 m-5000 m.

5. The method of claim 1, wherein the liquid-liquid separator is designed according to a gravitational settling principle; a main body of the liquid-liquid separator is a cylindrical cavity, which is vertically arranged; a lower end of the cylindrical cavity is provided with an inlet for the alkalified solution and an outlet of the bottom layer; an upper end of the cylindrical cavity is provided with an outlet of the top layer; an outer side of the cylindrical cavity is provided with a heat-exchanging interlayer for a heat-exchanging fluid to pass through; a lower part of the heat-exchanging interlayer is provided with a heat-exchanging fluid inlet, and an upper part of the heat-exchanging interlayer is provided with a heat-exchanging fluid outlet; and the liquid-liquid separator has an inner diameter of 1-20 cm and a height of 1-200 cm.

6. The method of claim 1, wherein the continuous stirred tank has a cylinder structure, and is vertically arranged; an interior of the continuous stirred tank is provided with a stirring blade, and an outer side of the continuous stirred tank is provided with a heat-exchanging jacket; a lower part of the continuous stirred tank is provided with a material inlet; an upper part of the continuous stirred tank is provided with a material outlet; a lower part of the heat-exchanging jacket is provided with a heat-exchanging fluid inlet, and an upper part of the heat-exchanging jacket is provided with a heat-exchanging fluid outlet; the material inlet is in pipeline connection with the liquid-liquid separator, and the material outlet is in pipeline connection with the second pump; and the continuous stirred tank has an inner diameter of 5-1000 cm and a height of 5-1000 cm.

7. The method of claim 1, wherein the first base is liquid ammonia, triethylamine, trimethylamine, tributylamine, diethylamine or N, N-diisopropylethylamine;
the second base is potassium carbonate, sodium carbonate, sodium bicarbonate, sodium hydroxide, potassium hydroxide or lithium hydroxide; and
the water-absorbing agent is selected from the group consisting of potassium carbonate, sodium carbonate, sodium bicarbonate, sodium hydroxide, potassium hydroxide, lithium hydroxide, sodium sulfate, magnesium sulfate, molecular sieve and a combination thereof.

8. A device for continuous recycling of an organic base, comprising:
a first pump;
a second pump;
a third pump;
a micro-mixer;
a micro-channel reactor;
a liquid-liquid separator; and
a continuous stirred tank;
wherein the micro-mixer, the micro-channel reactor, the liquid-liquid separator and the continuous stirred tank are connected sequentially through a pipeline; the first pump and the third pump are connected to the micro-mixer; the second pump is connected to the continuous stirred tank, and is configured to pump a first base from the continuous stirred tank to a first storage tank; the first pump is configured to pump a solution of a second base to the micro-mixer; the third pump is configured to pump a waste liquid containing the first base from a second storage tank to the micro-mixer; the micro-mixer is configured to mix the first base with the solution of the second base to obtain a mixture, and transfer the mixture to the micro-channel reactor for alkalization to obtain an alkalified solution; the liquid-liquid separator is configured to divide the alkalified solution into a top layer and a bottom layer; a lower part of the liquid-liquid separator is provided with a first outlet for discharging the bottom layer; and an upper part of the liquid-liquid separator is provided with a second outlet for discharging the top layer to the continuous stirred tank.

9. The device of claim 8, wherein the first pump, the second pump and the third pump are each a plunger pump for liquid transfer;
the micro-mixer is a composite plate-type micro-mixer composed of 15-18 rhombic microchannel mixing components connected in series; a fluid channel of each of the rhombic microchannel mixing components has a cross section of a first fluid channel of round or square; a size of the cross section (diameter or side length) is 100 μm-20 mm; a length of the first fluid channel is 1-100 cm; and an applicable flux is 1-3000 mL/min;
the micro-channel reactor is a tubular micro-reactor equipped with a static mixing component; a main body of the micro-channel reactor is a tubular cavity, and is configured as a second fluid channel; a first end of the tubular cavity is provided with a reactant material inlet, and a second end of the tubular cavity is provided with a reactant material outlet; a series of components in an double-cross shape is axially arranged in the tubular cavity; a first heat-exchanging fluid interlayer is provided outside the tubular cavity, and is configured for a first heat-exchanging fluid to pass through; a first end of the first heat-exchanging fluid interlayer is provided with a first heat-exchanging fluid outlet, and a second end of the first heat-exchanging fluid interlayer is provided with a first heat-exchanging fluid inlet; and a diameter of the second fluid channel is 100 μm-20 mm, and a length of the second fluid channel is 1 m-5000 m;
the liquid-liquid separator is designed according to a gravitational settling principle; a main body of the liquid-liquid separator is a cylindrical cavity, and is vertically arranged; a lower end of the cylindrical cavity is provided with an inlet for the alkalified solution, and the first outlet for discharging the bottom layer; an upper end of the cylindrical cavity is provided with the second outlet for discharging the top layer; an outer side of the cylindrical cavity is provided with a second heat-exchanging interlayer, and the second heat-exchanging fluid interlayer is configured for a second heat-exchanging fluid to pass through; a lower part of the second heat-exchanging fluid interlayer is provided with a second heat-exchanging fluid inlet, and an upper part of the second heat-exchanging fluid interlayer is provided with a second heat-exchanging fluid outlet; the liquid-liquid separator has an inner diameter of 1-20 cm and a height of 1-200 cm;
the continuous stirred tank is a cylinder, and is vertically placed; an interior of the continuous stirred tank is provided with a stirring blade, and an outer side of the continuous stirred tank is provided with a heat-exchanging jacket; a lower part of the continuous stirred tank is provided with a material inlet, and an upper part of the continuous stirred tank is provided with a material outlet; a lower part of the heat-exchanging jacket is provided with a third heat-exchanging fluid inlet, and an upper part of the heat-exchanging jacket is provided with a third heat-exchanging fluid outlet; the continuous stirred tank is filled with the water-absorbing agent; the material inlet is in pipeline connection with the liquid-liquid separator, and the material outlet is communicated with the second pump through the pipeline; and
the continuous stirred tank has an inner diameter of 5-1000 cm and a height of 5-1000 cm.
